(19) United States
(12) Patent Application Publication
(10) Pub. No.: US 2021/0076953 A1
(43) Pub. Date: Mar. 18, 2021

(54) METHODS AND SYSTEMS FOR PULSE TRANSIT TIME DETERMINATION

(71) Applicant: VITA-COURSE TECHNOLOGIES CO., LTD., Shenzhen (CN)

(72) Inventors: Zijian HUANG, Shenzhen (CN); Chuanmin WEI, Shenzhen (CN); Heng PENG, Shenzhen (CN); Ying LU, Shenzhen (CN); Jiwei ZHAO, Shenzhen (CN)

(73) Assignee: VITA-COURSE TECHNOLOGIES CO., LTD., Shenzhen (CN)

(21) Appl. No.: 17/106,213

(22) Filed: Nov. 30, 2020

Related U.S. Application Data
(63) Continuation of application No. PCT/CN2018/089542, filed on Jun. 1, 2018.

(51) Int. Cl. A61B 5/021; A61B 5/00

(52) U.S. Cl. CPC A61B 5/02125; A61B 5/7246; A61B 5/7267

(57) ABSTRACT

Methods and systems are provided for determining a cardiovascular parameter related to a cardiovascular system of a subject such as the pulse transit time (PTT). An exemplary method may include retrieving a photoplethysmogram (PPG) signal of a subject and determining a plurality of first parameters related to the PPG signal. The method may also include determining a second parameter of the subject. The second parameter may indicate a random effect of the subject. The method may further include determining the cardiovascular parameter based at least on the plurality of first parameters and the second parameter via a trained model.

### CROSS-REFERENCE TO RELATED APPLICATIONS

This application is a Continuation of International Application No. PCT/CN2018/089542, filed on Jun. 1, 2018, the entire contents of which are hereby incorporated by reference.

### TECHNICAL FIELD

The present disclosure generally relates to pulse transit time determination, and specifically relates to methods and systems for determining pulse transit time using a machine learning algorithm.

### BACKGROUND

Arterial pulse transit time (PTT) is the time that it takes a blood pressure wave to propagate along an artery from the heart to the periphery when the heart ejects stroke volume to the arteries. PTT may have a high correlation with cardiovascular characteristics such as systolic bold pressure and diastolic blood presser, and may be measured for diagnosing various types of cardiovascular and cerebrovascular disease. For example, PTT may server as a metric of arterial stiffness, and may be used for an estimation of trend in arterial blood pressure. Moreover, PTT monitoring may be valuable in the management of hypertension, in terms of assessing efficacy of a pharmacologic agent and titrating its dose.

In prior art, a measurement or determination of PTT of an individual relies on both a measurement of electrocardiographic (ECG) signals and a measurement of the photoplethysmogram (PPG) signals of that individual. In general, the measurement of PPG signals is relatively easy to be performed by, for example, using a single sensor (e.g., a pulse oximeter) wearing on the tip of the limb (such as a finger). However, the measurement of ECG signals is relatively complicated. For example, for measuring ECG signals, it is necessary to wear a number of electrode pads on multiple locations of the chest, hands, etc., which requires specific measuring equipment and is inconvenient in actual measurement operations. The synchronization between the measured PPG signals and ECG signals is also troublesome and may introduce additional errors. Therefore, there is a desire to provide method and system to determine the PTT of an individual more efficiently.

### SUMMARY

According to an aspect of the present disclosure, a method for determining a cardiovascular parameter (e.g., the pulse transit time (PTT)) related to a cardiovascular system of a subject is provided. The method may include retrieving a photoplethysmogram (PPG) signal of a subject and determining a plurality of first parameters related to the PPG signal. The method may also include determining a second parameter of the subject. The second parameter may indicate a random effect of the subject. The method may further include determining the cardiovascular parameter based at least on the plurality of first parameters and the second parameter via a trained model.

In some embodiments, the method may further include: selecting, from a plurality of pre-acquired PPG signals, at least one similar PPG signal by matching the PPG signal of the subject with the plurality of pre-acquired PPG signals; and determining the second parameter of the subject based at least on a second parameter associated with the at least one similar PPG signal. Each of the plurality of pre-acquired PPG signals may be associated with a second parameter.

In some embodiments, the plurality of second parameters associated with the plurality of pre-acquired PPG signals may satisfy a normal distribution or a generalized normal distribution.

In some embodiments, the determining a plurality of first parameters may include: retrieving at least one feature extracting mean; and determining at least some of the plurality of first parameters by extracting, via the at least one feature extracting mean, features based on at least one of the PPG signal, a first first-order derivative of the PPG signal, and a second-order derivative of the PPG signal.

In some embodiments, the method may further include training the model. The training the model may include determining a first plurality of candidate features. The first plurality of candidate features may include features associated with at least one of a PPG signal, a first-order derivative of the PPG signal, and a second-order derivative of the PPG signal. The training the model may also include obtaining a training dataset. The training dataset may include a plurality of standard PPG signals and a plurality of standard cardiovascular parameters (e.g., PTT) corresponding to the PPG signals. The training the model may further include: selecting, based on the training dataset, a second plurality of candidate features from the first plurality of candidate features using a feature selection routine; and determining a weight associated with each of the second plurality of candidate features by solving, based on the training dataset, a regression function. The regression function may include at least one variable associated with the second plurality of candidate features and at least one variable associated with the second parameter. By solving the regression function, a second parameter may be determined for each of the standard PPG signals. The training the model may also include: selecting, based on the determined weights, a plurality of target features from the second plurality of candidate features; and generating the model based on the plurality of target features and the weights thereof. The model may include a variable associated with the second parameter. The training the model may further include generating the at least one feature extracting mean according to the target features.

In some embodiments, the selecting a second plurality of candidate features from the first plurality of candidate features may include: determining, based on the training dataset, a plurality of correlations between the first plurality of candidate features. The second plurality of candidate features are selected based on the plurality of correlations.

In some embodiments, by solving the regression function based on the training dataset, one or more of the weights may be set to be zero.

In some embodiments, the determined second parameters of the standard PPG signals may satisfy a normal distribution or a generalized normal distribution.

In some embodiments, the regression function may be solved using an expectation maximization algorithm.

In some embodiments, the number of the first plurality of candidate features may range between 500 and 1000.

In some embodiments, the model may further include one or more variables associated with anthropometric character information of the subject. The method may further include determining, based on anthropometric characteristic information of the subject, one or more third parameters of the subject. The cardiovascular parameter may be determined based further on the one or more third parameters of the subject.

In some embodiments, the method may further include: generating, by a sensor, a raw PPG signal of the subject by detecting pulses of the subject for a predetermined time; and generating the PPG signal by preprocessing the raw PPG signal.

In some embodiments, the number of the plurality of first parameters may range between 30 and 150.

According to another aspect of the present disclosure, a system for determining a cardiovascular parameter (e.g., PTT) related to a cardiovascular system of a subject is provided. The system may include at least one processor and at least one storage device for storing instructions. The instructions, when executed by the at least one processor, may cause the system to retrieve a photoplethysmogram (PPG) signal of a subject and determine a plurality of first parameters related to the PPG signal. The system may be caused further to determine a second parameter of the subject. The second parameter may indicate a random effect of the subject. The system may be caused further to determine the cardiovascular parameter based at least on the plurality of first parameters and the second parameter via a trained model.

According yet to another aspect of the present disclosure, a system for determining a cardiovascular parameter (e.g., PTT) related to a cardiovascular system of a subject is provided. The system may include a PPG signal module, a first parameter module, a second parameter module, and a determination module. The PPG signal module may be configured to retrieve a photoplethysmogram (PPG) signal of a subject. The first parameter module may be configured to determine a plurality of first parameters related to the PPG signal. The second parameter module may be configured to determine a second parameter of the subject. The second parameter may indicate a random effect of the subject. The determination module may be configured to determine the cardiovascular parameter based at least on the plurality of first parameters and the second parameter via a trained model.

According yet to another aspect of the present disclosure, a non-transitory computer readable medium storing instructions is provided. The instructions, when executed by a processor, may cause the processor to execute operations for determining a cardiovascular parameter (e.g., PTT) related to a cardiovascular system of a subject. The operations may include retrieving a photoplethysmogram (PPG) signal of a subject and determining a plurality of first parameters related to the PPG signal. The operations may also include determining a second parameter of the subject. The second parameter may indicate a random effect of the subject. The operations may further include determining the cardiovascular parameter based at least on the plurality of first parameters and the second parameter via a trained model.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTIONS OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary system for determining a pulse transit time (PTT) of a subject according to some embodiments of the present disclosure;

FIG. 2 is a schematic diagram illustrating an exemplary computing device;

FIG. 3 is a schematic diagram illustrating an exemplary PTT determination device according to some embodiments of the present disclosure;

FIG. 4 is a flowchart illustrating an exemplary process of a PTT determination according to some embodiments of the present disclosure;

FIG. 5-A is a schematic diagram illustrating an exemplary test PPG signal;

FIG. 5-B is a schematic diagram illustrating an exemplary single-pulse PPG signal of the test PPG signal illustrated in FIG. 5-A;

FIG. 5-C is a schematic diagram illustrating the first-order derivative of the single-pulse PPG signal illustrated in FIG. 5-B;

FIG. 6 is a flowchart illustrating an exemplary process for determining a PTT based on a test PGG signal according to some embodiments of the present disclosure:

FIG. 7 is a schematic diagram illustrating an exemplary model training module according to some embodiments of the present disclosure; and

FIG. 8 is a flowchart illustrating an exemplary process for training a model for the PTT determination according to some embodiments of the present disclosure.

### DETAILED DESCRIPTION

Embodiments of the present disclosure provide methods and systems for performing a determination of a cardiovascular parameter (e.g., PTT) related to a cardiovascular system of a subject. The determination may be based on photoplethysmogram (PPG) signals of the subject and the random effect of the subject. Such a determination may be performed via a model, and the training of the model may involve one or more selections of features. The methods and systems are described by way of examples with reference to a determination of pulse transit time (PTT), and electrocardiographic (ECG) signals of the subject may not be involved in such a determination of PTT.

In the following detailed description, numerous specific details are set forth by way of examples in order to provide a thorough understanding of the relevant disclosure. However, it should be apparent to those skilled in the art that the present disclosure may be practiced without such details. In other instances, well known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “unit,” “module,” and/or “block” used herein are one method to distinguish different components, elements, parts, section or assembly of different level in ascending order. However, the terms may be displaced by another expression if they achieve the same purpose.

Generally, the word “module,” “sub-module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts.

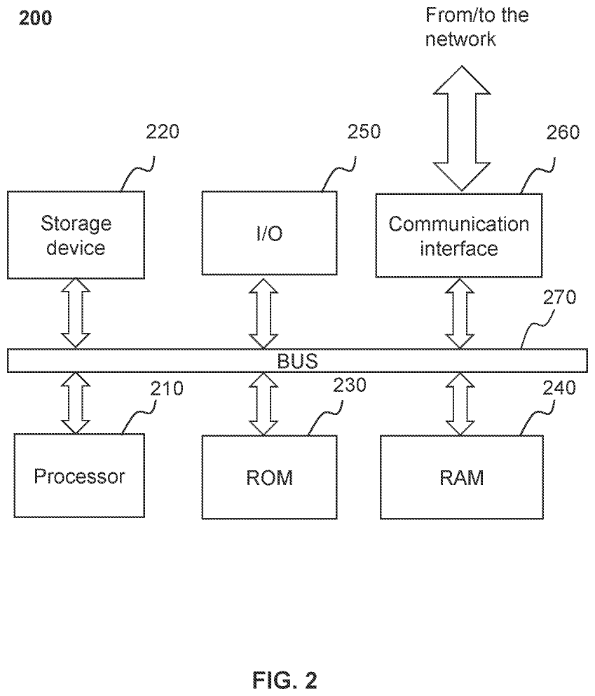

Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure.

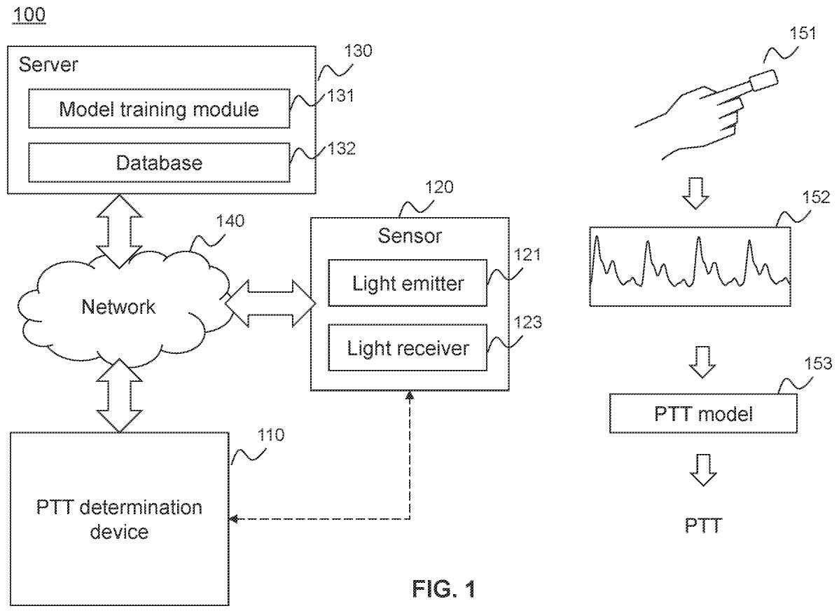

FIG. 1 is a schematic diagram illustrating an exemplary system for determining a pulse transit time (PTT) of a subject according to some embodiments of the present disclosure. A system 100 for determining the PTT of the subject may include a PTT determination device 110, a sensor 120, a server 130, and a network 140. The system 100 may further include additional devices or components in need.

The PTT determination device 110 may determine a PTT of a subject (e.g., a patient, a user) based on a photoplethysmogram (PPG) signal of the subject (or be referred to as a test PPG signal, e.g., the test PPG signal 152). The PTT determination device 110 may determine a plurality of first parameters related to the test PPG signal. Based on the plurality of first parameters and a second parameter of the subject indicating a random effect of the subject, the PTT determination device 110 may determine the PTT of the subject without using the ECG signal of the subject. In some embodiments, the server 130 may be implemented by the computing device illustrated in FIG. 2.

The PTT determination device 110 may input the plurality of first parameters and the second parameter into a PTT model 153, which may take the plurality of first parameters and the second parameter as at least part of the inputs, and determine a PTT as the output. For example, the PTT model 153 may include variables corresponding to the plurality of first parameters and the second parameter. In some embodiments, PTT model 153 may be a linear function including a plurality of coefficients (or weights) associated with its variables, and the determined PTT may be a weighted sum of the inputs.

The PTT determination device 110 may obtain the test PPG signal via the sensor 120 and/or from a storage device (e.g., the storage device 220 illustrated in FIG. 2) accessible to the network 140. For example, the PTT determination device 110 may obtain a raw PPG signal collected by the sensor 120 from the subject. The PTT determination device 110 may preprocess (e.g., noise reducing, smoothing) the raw PPG signal to generate the test PPG signal. As another example, the PTT determination device 110 may retrieve a pre-acquired PPG signal or a preprocessed raw PPG signal of the subject from the storage device. In some embodiments, the storage device may be included in the server 130 or communicatively connected to the server 130. For simplicity, unless otherwise stated, in the present disclosure, a raw PPG signal may generally refer to a PPG signal directly collected by a measuring device (e.g., the sensor 120) without further processing, and a PPG signal or a test PPG signal may generally refer to a preprocessed raw PPG signal.

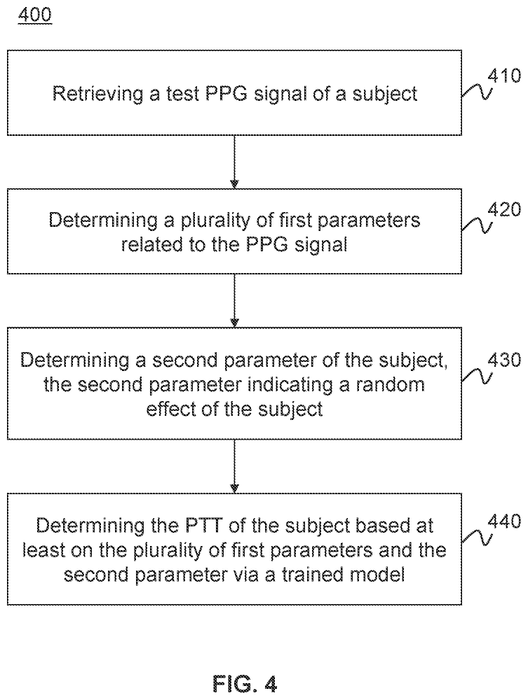

In some embodiments, the PTT determination device 110 may determine the plurality of first parameters by extracting features from data related to the test PPG signal. The data related to the test PPG signal may include at least one of the test PPG signal, the first-order derivative of the test PPG signal, or the second-order derivative of the test PPG signal. Detailed descriptions of the features and the plurality of first parameters are provided elsewhere in the present disclosure (e.g., in connection with FIGS. 4A to 4C).

The PTT determination device 110 may retrieve a pre-determined second parameter of the subject from a storage device (e.g., the storage device 220 illustrated in FIG. 2). Alternatively or additionally, the PTT determination device 110 may determine the second parameter in real time.

In some embodiments, the PTT determination device 110 may determine the PTT of the subject based further on one or more third parameters associated with anthropometric characteristic information of the subject. The PTT model 153 may further take the one or more third parameters as part of the inputs. For example, the PTT model 153 may also include one or more variables corresponding to the one or more third parameters. The anthropometric characteristic information of the subject may include long-term invariant information such as sex, race, and height (for adults), regularly changed information such as age, and short-term variant information such as weight, body fat rate, (and height for minors), or the like.

The PTT determination device 110 may determine the one or more third parameters based on anthropometric characteristic information of the subject. The PTT determination device 110 may retrieve the anthropometric characteristic information of the subject in various ways. For example, the PTT determination device 110 may provide an input mean (e.g., a touchscreen, a keyboard, a mouse, a microphone) allowing an operator (e.g., the subject, a technician) to input at least part of the anthropometric characteristic information of the subject. As another example, the system 100 may include at least one measuring device to obtain at least part of the anthropometric characteristic information (e.g., weight, height) by performing a corresponding measurement on the subject. The at least one measure device may transmit the obtained anthropometric characteristic information to the PTT determination device 110 via one or more cables or the network 140. As a further example, the PTT determination device 110 may retrieve pre-recorded anthropometric characteristic information of the subject from a database (e.g., the database 132) using identity information of the subject. Yet as another example, the PTT determination device 110 may analyze an image of the subject to determine at least part of anthropometric characteristic information of the subject (e.g., sex, race).

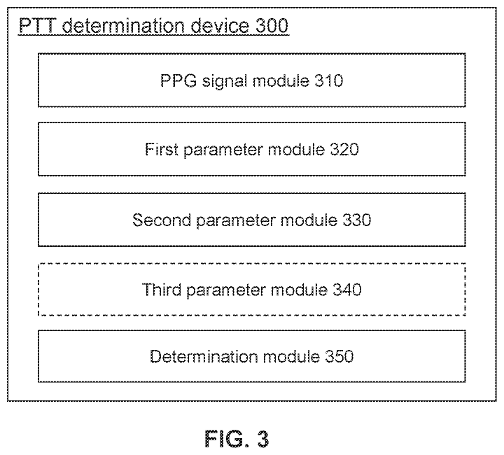

Detailed descriptions of the PTT determination device 110 and exemplary PTT determination process are provided elsewhere in the present disclosure (e.g., in connection with FIG. 3).

The sensor 120 may collect raw PPG signals from a subject. The sensor 120 may be placed on a limb (e.g., fingertip, wrist), the neck, an earlobe, etc., of the subject for sampling the raw PPG signals. In some embodiments, the sensor 120 may be photoelectric sensor and may include a light emitter 121 and a light receiver 123. The light emitter 121 may emit light to the subject. The light may penetrate through the subject or be reflected from the subject. The light receiver 123 may receive the reflected light or the penetrating light. The sensor 120 may detect a difference between the emitted light and the received light and generate a raw PPG signal therefrom. In some embodiments, the light emitter 121 may include a light emitting diode (LED) or a laser diode (LD), and the light receiver 123 may include a photo diode or an image sensor such as a complementary metal-oxide semiconductor (CMOS) image sensor (CIS). It may be noted that, the sensor 120 may be of any device capable of measuring a PPG signal of a subject, and is not limited to a photoelectric sensor.

In some embodiments, the PTT determination device 110 and the sensor 120 may communicate with each other via one or more cables (e.g., broken arrow illustrated in FIG. 1) or the network 140. For example, the sensor 120 may be a photoelectric sensor (e.g., included in a pulse oximeter 151) and the PTT determination device 110 may be a terminal device. The terminal device may be a personal computer (PC), a server, a mobile computing device, a wearable computing device, etc. For example, the PTT determination device 110 may be a mobile computing device (e.g., a mobile phone, a tablet computer) and may communicate with the sensor 120 via the network 140 (e.g., a Wi-Fi network, a Bluetooth™ network).

In some embodiments, the sensor 120 may be included in the PTT determination device 110. For example, the PTT determination device 110 may be a wearable computing device such as a smart bracelet, a smart band, a smart watch, a Virtual Reality (VR) equipment, etc. When a subject is wearing the PTT determination device 110, the sensor 120 may be at a location proper for sampling a raw PPG signal of the subject. The PTT determination device 110 (e.g., a smart watch) may include a screen for displaying the determined PTT of the subject. Alternatively or additionally, the PTT determination device 110 may transmit (e.g., via the network 140) the determined PTT to a device including a screen (e.g., a mobile phone, a television, a computer, a virtual reality equipment) or a projector for display.

In some embodiments, the sensor 120 may transmit a raw PPG signal to the PTT determination device 110, and the PTT determination device 110 may preprocess (e.g., noise reducing, smoothing) the raw PPG signal to generate a test PPG signal for the PTT determination. Alternatively or additionally, the sensor 120 may include logic circuits for preprocessing a raw PPG signal, and transmit the preprocessed PPG signal to the PTT determination device 110. The PTT determination device 110 may directly perform the PTT determination on the received PPG signal without further processing the raw PPG signal.

In some embodiments, the PTT determination device 110 may transmit a control signal to the sensor 120 for controlling the sampling of the raw PPG signal.

The server 130 may be local or remote. The server 130 may include a model training module 131 and a database 132. The model training module 131 may retrieve a training dataset from the database 132 and train the PTT model 153 using the training dataset. The PTT determination device 110 may retrieve the trained PTT model 153 from the server via the network 140 and operate the retrieved PTT model 153 for determining the PTT of the subject. Alternatively, the PTT determination device 110 may transmit the plurality of first parameters, the second parameter (optional), and the one or more third parameters (optional) to the server 130 via the network 140. The server 130 may operate the trained PTT model 153 to determine the PTT of the subject and then transmit the determined PTT to the PTT determination device 110.

The server 130 may be a single server or a server group. For example, the server 130 may be a single server and both the model training module 131 and the data based 132 may be included in such a single server. As another example, the server 130 may be a server group. The model training module 131 may be implemented by one or more servers of the server group and the database 132 may be implemented by another or some other servers of the server group. Such a server group may be centralized, or distributed (e.g., the server 130 may be a distributed system). In some embodiments, the server 130 may be implemented by the computing device illustrated in FIG. 2.

In some embodiments, the database 132 may be implemented by a storage device (e.g., the storage device 220 illustrated in FIG. 2) or a group of storage devices. The database 132 may include a plurality of pre-acquired PPG signals (or be referred to as standard PPG signals). Each of the standard PPG signals may be associated with a standard PTT, which may be obtained by performing a PTT measurement or determination routine in the art on the subject associated with the corresponding standard PPG signal. For example, for determining a standard PTT, a PGG signal sampling operation and an ECG signal sampling operation may be performed on a subject simultaneously. The standard PTT may be determined based on the collected PGG signal and the collected ECG signal, and then be stored in the database 132. The collected PPG signal may also be stored in the database 132 as the standard PPG associated with the standard PTT.

The database 132 may further include a plurality of second parameters, each of which is associated with a standard PPG signal. Each of the plurality of the second parameters may indicate a random effect of the subject associated with the corresponding standard PPG signal. The “random effect” in statistics refers to a subject-specific effect of a subject with respect to the population-average. In the present disclosure, the “population-average” may be viewed as a PTT determined considering only the known features of the PGG signal and/or the subject (e.g., features extracted for determining PTT, features extracted for train the model), the “random effect” may be viewed as a subject-specific bias considering unknown features of the PGG signal and/or the subject. The second parameter may be such a bias, or be used to determine such a bias.

In some embodiments, a second parameter of a first subject may also be used for determining the PTT of a second subject if the physiological characteristics (at least part of which is/are associated with the cardiovascular system) of the second subject are determined (e.g., by the PTT determination device 110 or the server 130) to be similar to those of the first subject. For example, if the PTT determination device 110 (or the server 130) determines that the test PPG signal of the second subject is (e.g., based on a matching algorithm or a matching strategy) similar to the test PPG signal of the first subject, and/or the anthropometric characteristic information of the second subject is similar to the anthropometric characteristic information of the first subject, the PTT determination device 110 may treat the second parameter of the first subject as the second parameter of the second subject or determine the second parameter of the second subject based on the second parameter of the first subject.

In the present disclosure, the model for determining the PTT (or any other cardiovascular parameter) may be considered as formed by two parts, the first part may be used for determining a “population-average” on PTT and may take the first plurality of parameters (and the one or more third parameters in some embodiments) as at least some of its inputs, the second part may be used as for determining a “subject-specific effect” (or the “random effect”) of the subject on PTT and may take the second parameter as its input or one of its inputs. The determined PTT of a subject may be viewed a result of the “population-average” affected by the “random effect” of the subject. In some embodiments, the determined PTT of the subject is the sum of the “population-average” determined by the first part of the model and the “random effect” (or bias) determined by the second part of the model.

In some embodiments, the second parameter itself may be the subject-specific bias. The second parameters associated with the standard PPG signals may satisfy a certain distribution, such as a normal distribution, a generalized normal distribution (e.g., an exponential power distribution, a skew normal distribution). In some embodiments, the second parameters in the dataset 132 may be determined during the training of the PTT model 153, e.g., by the model training module 131. The determined second parameter may be stored in the database 132 and associated with the corresponding standard PPG signal.

In some embodiments, the server 130 or the database 132 may be referred to as a datacenter or a data warehouse. Related techniques may be adopted to construct, operate, update, and/or maintain the server 130 or the database 132.

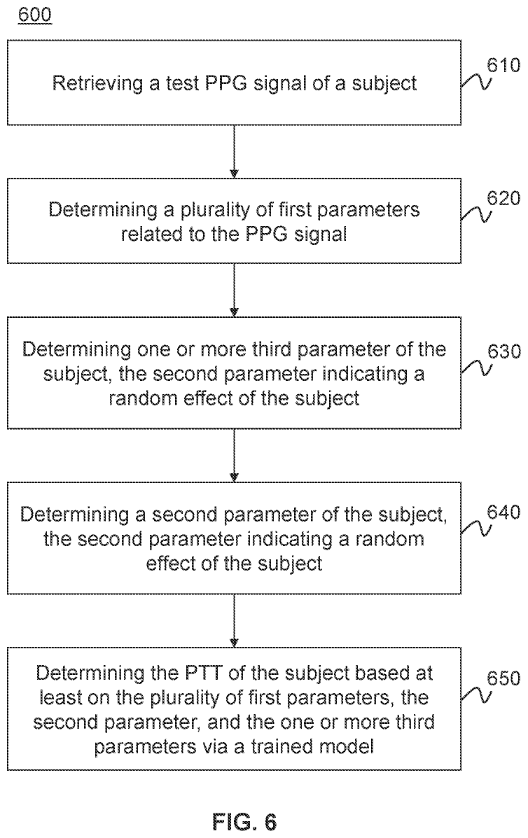

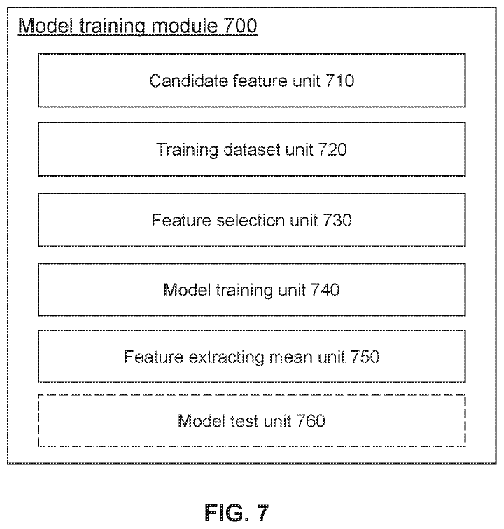

The model training module 153 may retrieve at least some of standard PPG signals and the corresponding standard PTTs to form the training dataset of the PTT model 153. Detailed descriptions of the model training module 131 and the training of the PTT model 153 are provided elsewhere in the present disclosure (e.g., in connection with FIGS. 6 and 7).

In some embodiments, the PTT model 153 may further take the aforementioned one or more third parameters associated with the anthropometric characteristic information of the subject as inputs. The database 132 may also include anthropometric characteristic information of a subject associated with each of the standard PPG signals. The model training module 131 may further retrieve the anthropometric characteristic information associated with the at least some of standard PPG signals to form the training dataset of the PTT model 153.

The network 140 may include any suitable network that can facilitate the exchange of information and/or data for the system 100. In some embodiments, one or more components of the system 100 (e.g., PTT determination device 110, the sensor 120, the server 130) may communicate information and/or data with one or more other components of the system 100 via the network 140. For example, the PTT determination device 110 may obtain raw PPG data from the sensor 140 via the network 140. The network 140 may be and/or include a public network (e.g., the Internet), a private network (e.g., a local area network (LAN), a wide area network (WAN)), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 140 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 140 may include one or more network access points. For example, the network 140 may include wired and/or wireless network access points such as base stations and/or internet exchange points through which one or more components of the system 100 may be connected to the network 140 to exchange data and/or information.

For convenience of description and demonstration purposes, the present disclosure is described herein by way of example with reference to the PTT determination. However, it is understood that the principle of the present disclosure may be applied to a determination of an alternative cardiovascular parameter. For example, the system 100 (or the PTT determination device 110) may be configured to determine, based on data related to the PPG signal, one or more cardiovascular parameters of the subject other than the PTT, such as the pulse wave velocity (PWV), the pulse wave amplitude (PWA), the systolic blood pressure, the diastolic blood pressure, the pulse pressure, etc. For example, for determining an alternative cardiovascular parameter such as the PWV of the subject, the database 132 may include a standard PWV associated with each of the standard PPG signals, and the model training module 131 may retrieve a training dataset including the standard PWVs and the corresponding standard PPG signals to train a corresponding model (or be referred to as a PWV model). Via the PWV model, the PTT determination device 110 (which may not be configured to determine the PTT now, but the name is retained for convenience of description) may determine the PWV of a subject based on a test PPG signal of the subject (e.g., obtained via the sensor 120).

Similarly, the system 100 may be configured to determine one or more other cardiovascular parameters. Unless otherwise stated, the “PTT” described in the present disclosure may be replaced by any other cardiovascular parameter mentioned or not mentioned in the present disclosure.

In some embodiments, the model training module 131 may train a plurality of models, each of which is trained to determine a corresponding cardiovascular parameter based on a PPG signal. The PTT determination device 110 may determine, via the plurality of models, the corresponding cardiovascular parameters (including or not including the PTT) based on the same test PPG signal at the same time. For example, the PTT determination device 110 may determine both of the systolic blood pressure and the diastolic blood pressure based on the same test PPG signal.

In some embodiments, the PTT determination device 110 may further determine one or more cardiovascular parameters based on the PTT determined by the PTT model 153 (or any other cardiovascular parameter determined by a model trained by the model training module 131). For example, the PTT determination device 110 may determine the PWV based on the determined PTT, instead of using a model trained by the model training module 131 for determining the PWV.

It may be noted that, the above description about the system 100 is only for illustration purposes, and is not intended to be limiting. It is understood that, after learning the major concept of the present disclosure, a person of ordinary skill in the art may alter the system 100 in an uncreative manner. The alteration may include combining and/or splitting modules or devices, adding or removing optional modules or devices, etc. All such modifications are within the scope of the present disclosure.

For example, the PTT determination device 110 and the sensor 120 may be integrated in a wearable device, which may be directly worn by a user and capable of performing a determination of PTT (and/or any other cardiovascular parameter such as blood pressure) periodically, in response to an instruction of the user, or according to a predetermined measurement plan.

As another example, the PTT determination device 110 may be integrated in the server 130. The sensor 120 may serve as a terminal device (e.g., an oximeter capable of accessing to the network 140), and may transmit a raw PPG signal or a preprocessed PPG signal of a user to the server 130 via the network 140. The server 130 may receive the raw PPG signal or the preprocessed PPG signal and perform a determination of PTT (and/or any other cardiovascular parameter) based on the received signal. The determined PTT may be transmitted to the sensor 120 and be presented to the user via, for example, a display (e.g., a touch screen) of the sensor 120. In some embodiments, the sensor 120 may further receive anthropometric characteristic information inputted by the user (e.g., via the touch screen of the sensor 120) and transmit the received anthropometric characteristic information to the server 130 for the PTT determination. Alternatively or additionally, the user may transmit the anthropometric characteristic information to the server 130 via another device capable of accessing the network 140, such as a mobile phone, a PC, and an online measuring device.

FIG. 2 is a schematic diagram illustrating an exemplary computing device. Computing device 200 may be configured to implement the PTT determination device 110, the server 130, and/or any other component of the system 100. The computing device may perform one or more operations disclosed in the present disclosure. The computing device 200 may include a bus 270, a processor 210, a read only memory (ROM) 230, a random access memory (RAM) 240, a storage device 220 (e.g., massive storage device such as a hard disk, an optical disk, a solid-state disk, a memory card, etc.), an input/output (I/O) port 250, and a communication interface 260. It may be noted that, the architecture of the computing device 200 illustrated in FIG. 2 is only for demonstration purposes, and not intended to be limiting. The computing device 200 may be any device capable of performing a computation.

In some embodiments, the computing device 200 may be a single device. Alternatively, the computing device 200 may include a plurality of computing devices having a same or similar architecture as illustrated in FIG. 2, and one or more components of the computing device 200 may be implemented by one or more of the plurality of computing devices.

The bus 270 may couple various components of computing device 200 and facilitate transferring of data and/or information between them. The bus 270 may have any bus structure in the art. For example, the bus 270 may be or may include a memory bus and/or a peripheral bus.

The 1/O port 250 may allow a transferring of data and/or information between the bus 270 and one or more peripheral device (e.g., one or more cameras 220, one or more input devices (e.g., a keyboard, a mouse, a joystick, a microphone), one or more output devices (e.g., a display, a loudspeaker, a printer)). The 1/O port 250 may include a USB port, a COM port, a PS/2 port, an HDMI port, a VGA port, a video cable socket such as an RCA sockets and a Mini-DIN socket, a coaxial cable port (e.g., for implementing a POC technique), or the like, or a combination thereof. In some embodiments, the I/O port 250 may be coupled to the sensor 120 illustrated in FIG. 1 for transferring a raw PPG signal or a preprocessed PPG signal from the sensor 120 to the bus 270, which may be further transferred to the storage device 220, the RAM 240, or the Processor 210.

The communication interface 260 may allow a transferring of data and/or information between the network 140 and the bus 270. For example, the communication interface 260 may be or may include a network interface card (NIC), a Bluetooth™ module, an NFC module, etc. In some embodiments, the communication interface 260 may communicate the sensor 120 illustrated in FIG. 1 via the network 140 for transferring a raw PPG signal or a preprocessed PPG signal from the sensor 120 to the bus 270.

The ROM 230, the RAM 240, and/or the storage device 220 may be configured to store computer readable instructions that may be executed by the processor 210. The RAM 240, and/or the storage device 220 may store date and/or information obtained from a peripheral device (e.g., one or more cameras 220) and/or the network 150/260. The RAM 240, and/or the storage device 220 may also store date and/or information generated by the processor 210 during the execution of the instruction. In some embodiments, the storage device 220 may implement the database 132 for storing, for example, standard PPG signals, standard PTTs (and/or any other cardiovascular parameters), second parameters, and/or anthropometric characteristic information.

The processor 210 may include any processor in the art configured to execute computer readable instructions (e.g., stored in the ROM 230, the RAM 240, and/or the storage device 220), so as to perform one or more operations or implement one or more modules/units disclosed in the present disclosure.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed. In some embodiments, the computer may be a mobile computing device or a wearable computing device.

FIG. 3 is a schematic diagram illustrating an exemplary PTT determination device according to some embodiments of the present disclosure. PTT determination device 300 is an example of the PTT determination device 100, which may be configured to determine the PTT (and/or any other cardiovascular parameter) of a subject based on the PPG signal of the subject. The PTT determination device 300 may include a PPG signal model 310, a first parameter module 320, a second parameter module 330, and a determination module 350. In some embodiments, the PTT determination device may further include a third parameter module 340.

The PTT determination device 300 and the modules thereof may be implemented by the computing device 200 illustrated by FIG. 2.

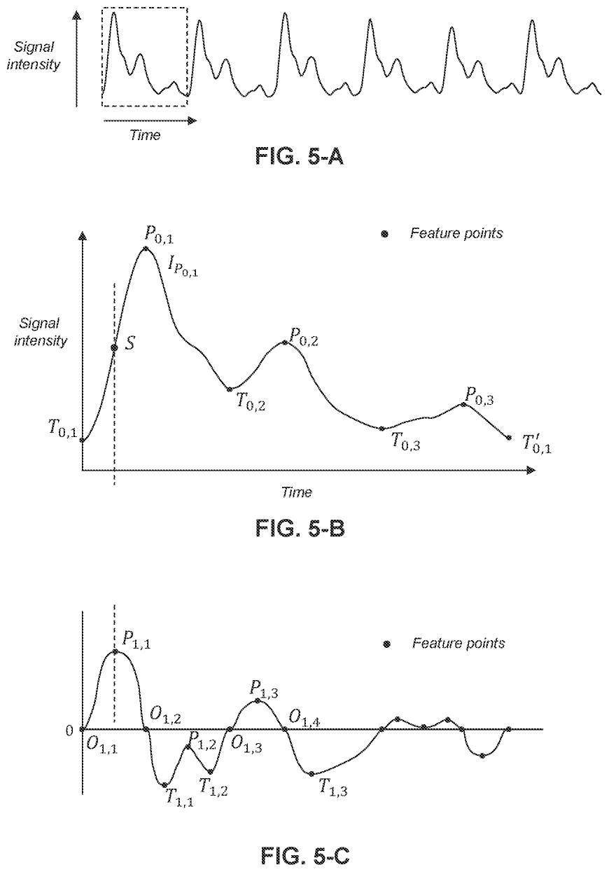

The PPG signal module 310 may be configured to retrieve a test PPG signal of the subject. An exemplary test PPG signal is illustrated in FIG. 5-A. In some embodiment, the PPG signal module 310 may retrieve a raw PPG signal from the sensor 120, and preprocess (e.g., noise reducing, smoothing) the raw PPG signal to generate the test PPG signal.

The first parameter module 320 may be configured to determine a plurality of first parameters related to the test PPG signal. In some embodiments, the first parameter module 320 may generate at least one the first-order derivative of the test PPG signal and the second-order test derivative of the PPG signal. The first parameter module 320 may determine the plurality of first parameters by extracting features from at least one of the test PPG signal, the first-order derivative of the test PPG, or the second-order derivate of the test PPG signal. In the present disclosure, features extracted from data related to a test PPG signal may be referred to as first features.

The second parameter module 330 may be configured to determine a second parameter of the subject, which may indicate a random effect of the subject. In some embodiments, the second parameter module 330 may retrieve a pre-determined second parameter of the subject from a storage device (e.g., the database 132, the storage device 220). Alternatively or additionally, the second parameter module 330 may perform a matching between the test PPG signal and pre-acquired PPG signals (e.g., the aforementioned standard PPG signals) in a database (e.g., the database 132). Each of the pre-acquired PPG signals may associated with a determined second parameter. The second parameter module 330 may determine the second parameter of the subject based on the matching result.

The third parameter module 340 may be configured to determine one or more third parameters related to the anthropometric characteristic information of the subject. The third parameter module 340 may retrieve the anthropometric characteristic information of the subject from a storage device (e.g., the database 132, the storage device 220) or from one or more measuring devices. Alternatively or additionally, the third parameter module 340 may receive the anthropometric characteristic information via an input mean provided by the PTT determination device 300 for a user (e.g., the subject) to input the anthropometric characteristic information of the subject. The third parameter module 340 may determine the one or more third parameters based on the anthropometric characteristic information of the subject.

The determination module 350 may be configured to determine the PTT of the subject based at least on the plurality of first parameters and the second parameter via a trained model. In some embodiments, the determination module 350 may determine the PTT based further on the one or more third parameters via the trained model.

The PTT determination device 300 may determine the PTT of the subject via a process (e.g., process 400) described in connection with FIG. 4 or a process (e.g., process 400) described in connection with FIG. 6.

It may be noted that, the above descriptions about the PTT determination device 300 are only for illustration purposes, and are not intended to limit the present disclosure. It is understandable that, after learning the major concept and the mechanism of the present disclosure, a person of ordinary skill in the art may alter the PTT determination device 300 in an uncreative manner. The alteration may include combining and/or splitting modules or sub-modules, adding or removing optional modules or sub-modules, etc. All such modifications are within the scope of the present disclosure.

FIG. 4 is a flowchart illustrating an exemplary process of a PTT determination according to some embodiments of the present disclosure. Process 400 may be performed to determine the PTT of a subject based on a test PPG signal of the subject. In some embodiments, one or more operations of the process 400 illustrated in FIG. 4 may be implemented in the PTT determination device 300 illustrated in FIG. 3. For example, the process 400 illustrated in FIG. 4 may be stored in a storage device (e.g., the storage device 220) in the form of instructions, and invoked and/or executed by at least one processor (e.g., the processor 210 of the computing device 200 as illustrated in FIG. 2).

In 410, the PPG signal module 310 may retrieve a test PPG signal of a subject. The PPG signal module 310 may retrieve the test PPG signal from, for example, a storage device (e.g., the storage device 220) or the sensor 120. Alternatively or additionally, the PPG signal module 310 may retrieve a raw PPG signal from a storage device or the sensor 120 and preprocess the raw PPG signal to generate the test PPG signal.

In some embodiments, the preprocessing of the raw PPG signal by the PPG signal module 310 may include a noise reduction of the raw PPG signal. The PPG signal module 310 may perform the noise reduction the raw PPG signal via any noise reduction routine in the art, such as filtering, adaptive filtering, polynomial fitting, wavelet transformation, motion compensation, a fractal based technique, or the like, or a combination thereof.

Refer to FIG. 5-A. FIG. 5-A is a schematic diagram illustrating an exemplary test PPG signal. A test PPG signal may be generated (e.g., by the PPG signal module 310 or the sensor 120) by preprocessing a raw PPG signal collected, for example, by the sensor 120 within a predetermined time window (e.g., 10 seconds, 20 seconds, 30 seconds, or any other proper time interval). The test PPG signal may include PPG signals of a plurality pulses detected during the predetermined time window. A PPG signal of a single pulse (e.g., the dashed box as illustrated in FIG. 5-A) is further illustrated in FIG. 5-B.

Refer back to FIG. 4. In 420, the first parameter module 320 may determine a plurality of first parameters related to the test PPG signal. The first parameters module 320 may determine the plurality of first parameters by extracting first features from data related to the test PPG signal. The determined first parameters may serve as inputs of the model (e.g., the PTT model 153) for determining the PTT of the subject.

The data related to the test PPG signal may include at least one of the test PPG signal itself, the first-order derivative of the test PPG signal (e.g., as illustrated in FIG. 5-C), and the second-order derivative of the test PPG signal (not shown). The first-order derivative of the test PPG signal is the derivative of the test PPG signal, and the second-order derivative of the test PPG signal is the derivative of the first-order derivative of the test PPG signal. In some embodiments, the first parameter module 320 may determine at least one of the first-order derivative of the test PPG signal or second-order derivative of the test PPG signal, and determine at least some of the plurality of first parameters by extracting first features based on the test PPG signal, the first-order derivative of the test PPG signal, and/or second-order derivative of the test PPG signal. A test PPG signal or a derivative (first-order, second-order, or a higher order) of a test PPG signal from which the first parameter module 320 extracts first features may also be referred to as a feature source.

For demonstration purposes, first features extracted by the first parameter module 320 are described in connection with FIGS. 5-B and 5-C. Refer to FIGS. 5-B and 5-C. FIG. 5-B is a schematic diagram illustrating an exemplary single-pulse PPG signal of the test PPG signal illustrated in FIG. 5-A. A single-pulse PPG signal may be a PPG signal corresponding to a single pulse, and may include a plurality of peaks (e.g., P_{0,1}, P_{0,2}, and P_{0,3}) and troughs (e.g., T_{0,1}, T_{0,2}, and T_{0,3}).

In some embodiments, a PPG signal of a single pulse collected by the sensor 120 may further include one or more zero-crossings (not show). For example, the zero value may be predetermined as the mean of the maximum value and the minimum value of a single-pulse PPG signal, the mean of the maximum value and the minimum value of the entire test PPG signal, or the mean of the maximum values and minimum values of all the single-pulse PPG signals included in the test PPG signal. In such cases, multiple zero-crossings may present in a single-pulse PPG signal. As another example, the zero value may be predetermined as the intensity value of the minimum value of the PPG signal of the current pulse or the minimum value of the entire test PPG signal. In such cases, a single zero-crossing, which is also a trough, may present in a single-pulse PPG signal or a test PPG signal. Alternatively, the entire PPG signal may be above zero and no zero-crossing may present in the PPG signal.

In some embodiments, whether the PPG signal has the zero value may be determined by the configuration (hardware or software) of the sensor 120, and/or the preprocessing routine applied on the raw PPG signal to generate the test PPG signal.

As illustrated in FIG. 5-B, a PPG signal of a single pulse may include three peaks P_{0,1}, P_{0,2}, and P_{0,3} and three troughs T_{0,1}, T_{0,2}, and T_{0,3}. The trough T_{0,1} may be the minimum point of the PPG signal of the current pulse and may be referred to as the primary trough or the first trough. The peak P_{0,1} may be the maximum point of the PPG signal of the current pulse and may be referred to as the primary peak or the first peak. A starting point of a PPG signal of a single pulse may be the primary trough T_{0,1} of the single pulse and the end point of the PPG signal may be the primary trough T′_{0,1} or the next pulse.

In accordance with the cardiovascular condition of a subject, a single-pulse PPG signal of the subject may be different from the one illustrated in FIG. 5-B. For example, additional peak(s) and/or trough(s) may present in a single-pulse PPG signal. As another example, a relative intensity value and/or a relative timestamp of a peak or a trough with respect to another peak or trough may vary.

FIG. 5-C is a schematic diagram illustrating the first-order derivative of the single-pulse PPG signal illustrated in FIG. 5-B. The first-order derivative of the single-pulse PPG signal may include a plurality of peaks (e.g., P_{1,1}, P_{1,2}, and P_{1,3}), troughs (e.g., T_{1,1}, T_{1,2}, and T_{1,3}), and zero-crossings (e.g., O_{1,1}, O_{1,2}, O_{1,3}, and O_{1,4}), according to the waveform of the single-pulse PPG signal. The second-order derivative of the single-pulse PPG signal (not shown) may also include a plurality of peaks, troughs, and zero-crossings, according to the waveform of the first-order derivative of the single-pulse PPG signal.

Refer back to FIG. 4. The first parameter 320 may determine one or more feature points on a feature source (e.g., a test PPG signal, the first-order derivative of the test PPG signal, and/or the second-order derivative of the test PPG signal), which may include but not limited to peaks, troughs, and zero-crossings (if any) of the feature source. In some embodiments, a feature point determined on a first feature source (e.g., the test PPG signal) may correspond to a peak, trough, or zero-crossing (if any) of a second feature source (e.g., the first-order derivative of the test PPG signal). For example, the feature point S determined on the test PPG signal illustrated in FIG. 5-B may correspond to the peak P_{1,1} of the first-order derivative of the test PPG signal illustrated in FIG. 5-C.

A feature point may have a plurality of attributes, such as an intensity value and a timestamp. The first parameter module 320 may extract first features from the feature source based on one or more attributes of at least some of the plurality of feature points thereof.

As a feature source may include a plurality of segments (e.g., a single-pulse PPG signal, the first/second-order derivative of a single-pulse PPG signal), each of which may correspond to a single pulse, the first parameter module 320 may extract a same set of first features from each segment of the plurality of segments, and obtain a plurality of first preliminary parameters for each segment correspondingly. A first preliminary parameter may be a value obtained by extracting a certain first feature from a single segment. Based on first preliminary parameters corresponding to a same first feature, the first parameter module 320 may determine a first parameter corresponding to that first feature. For example, a first parameter may be a mean, a median, a weighted mean, a mode (e.g., via a histogram based approach), etc., of the corresponding preliminary parameters.

For convenience of description, a specific first feature extracted by the first parameter model 320 in the present disclosure may be described with respect to a single segment of the corresponding feature source. However, it is understood that, for extracting such a first feature, in some embodiments, the first parameter model 320 may extract the same first feature from each segment of the corresponding feature source, and determine a first parameter as the extraction result based on the obtained first preliminary parameters. For example, for extracting a first feature described as “the intensity value of the first trough (e.g., T_{0,1}) of a single-pulse PPG signal”, the first parameter model 320 may acquire an intensity value of a feature point representing the first trough from each single-pulse PPG signal of a test PPG signal. The acquired intensity values may serve as the aforementioned first preliminary parameters. The first parameter model 320 may determine, for example, a mean, a median, a mode, etc., of the acquired intensity values as the first parameter corresponding to the first feature. Such a feature extracting manner may be referred to as a group-specific feature extracting manner.

A first feature extracted by the first parameter module 320 may be related to a single feature point or related to multiple feature points. For a first feature related to multiple feature points, the multiple feature point may be included in the same segment, or be respectively included in corresponding segments of different feature sources (e.g., a single-pulse PPG signal and the second-order derivative of the single-pulse PPG signal). In some embodiments, first features to be extracted by the first parameter module 320 may include but not limited to: the intensity value of the first trough (e.g., T_{0,1}) of a single-pulse PPG signal, the intensity value of the first peak (e.g., P_{0,1}) of a single-pulse PPG signal, the intensity value of the first peak (e.g., P_{1,1}) of the first-order derivate of a single-pulse PPG signal, a time interval between the third zero-crossing (e.g., O_{1,3}) and the fourth zero-crossing (e.g., O_{1,4}) of the first-order derivate of a single-pulse PPG signal, a ratio of the intensity value of the second trough (e.g., T_{0,2}) of a single-pulse PPG signal to the intensity value of the second peak (not shown) of the second-order derivative of the single-pulse PPG signal, etc. It may be noted that the above first features are only provided for demonstration purposes and not intended to be limiting.

The first parameter module 320 may extract M first features (M is a positive integer larger than 2) from data related to the test PPG signal for determining the PTT. Correspondingly, the first parameter module 320 may determine M first parameters. It is understood that, the number of first features extracted by the first parameter module 320 may be changed in need. In some embodiments, a value range of M may be [30, 150]. In some specific embodiments, a value range of M may be [40, 80]. In some more specific embodiments, a value range of M may be [50, 70].

In some embodiments, the first parameter module 320 may retrieve at least one first feature extracting mean and extract the first features based on the at least one first feature extracting mean. The first feature extracting mean may be in the form of, for example, a look-up table, a feature extracting model (e.g., including one or more functions), or the like, or a combination thereof. The first parameter module 320 may retrieve the at least one first feature extracting mean from a storage device (e.g., the storage device 220) or the server 130.

In some embodiments, the first feature extracting mean may include a look-up table including a plurality of items, each of which represents an association between a first feature to be extracted and the corresponding feature source. According to the look-up table, the first parameter module 320 may extract first features recorded in the look-up table from the associated feature source. The first parameter module 320 may include models or functions for performing the feature extraction. Alternatively or additionally, the first parameter module 320 may retrieve the models or functions from a storage device (e.g., the storage device 220) or the server 130.

In some embodiments, the first feature extracting mean may include a feature extracting model. By operating the feature extracting model on a corresponding feature source, the first parameter module 320 may extract one or more corresponding first features from the feature source and thereby obtain one or more corresponding first parameters. In some embodiments, the first feature extracting mean may be an advanced feature extracting model. The advanced feature extracting model may include all the information, model(s), and function(s) needed for determining the first parameters. By operating the advanced feature extracting model, the first parameter module 320 may generate the first-order derivative and/or the second-order derivative of the test PPG signal, and extract first features from the test PPG signal, the first-order derivate of the test PPG signal, and/or the second-order derivate of the test PPG signal.

In some embodiments, the first parameter module 320 may retrieve a plurality of first feature extracting means for determining corresponding cardiovascular parameters. Based on the retrieved first feature extracting means, the first parameter module 320 may determine a plurality of groups of first parameters (or be referred to as first parameter groups). Each of the first parameter groups is for determining a corresponding cardiovascular parameter. When a same first feature is needed for determining multiple cardiovascular parameters, the first parameter module 320 may extract the first feature from the test PPG signal for once (e.g., the corresponding first feature extracting means include or are look-up tables) and the obtained first parameter may be shared by the corresponding first parameter groups. Alternatively, the first parameter module 320 may extract the first feature from the test PPG signal for multiple times (e.g., the corresponding first feature extracting means include or are feature extracting models), and at each time a first parameter is determined for a corresponding first parameter group.

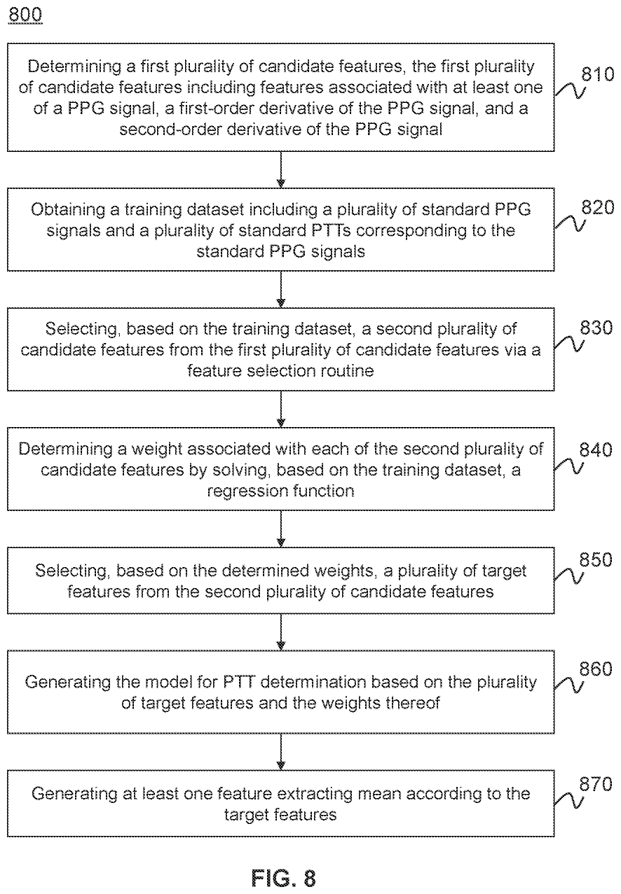

In some embodiments, the first feature extracting mean may be generated during the training of the model, the descriptions of which may be found elsewhere in the present disclosure (e.g., in connection with FIG. 8).

In 430, the second parameter module 330 may determine a second parameter of the subject. The second parameter may indicate a random effect of the subject, and may also serve as an input of the model for determining the PTT of the subject. In some embodiments, the second parameter may be predetermined (e.g., by the second parameter module 330) based on a pre-acquired test PPG signal of the subject and the corresponding PTT of the subject. The PTT of the subject may be obtained by applying a PTT measurement or determination routine in the art on the pre-acquired test PPG signal (e.g., via a determination routine based on simultaneously collected PPG signal and ECG signal of the subject). The first parameter module 320 may determine a plurality of first parameters by extracting first features form the pre-acquired test PPG signal. The second parameter module 330 may then determine the second parameter of the subject based on the PTT of the subject, the plurality of first parameters, and the model for determining the PTT (e.g., the PTT model 153). For example, the model may be in the form of y=f(X, α), wherein X refers to the plurality of first parameters, a refers to the second parameter, and y may refers to the determined PTT. The model may be rewrite in the form of α=f′(y, X). By inputting the standard PTT and the plurality of first parameters into the model, the a may be determined as the output.

The determined second parameter may be stored in a storage device (e.g., the storage device 220, or the database 132), and be used for subsequent PTT determinations of the subject.

In some embodiments, the second parameter module 330 may determine the second parameter of the subject in real-time. For example, the second parameter module 330 may perform a matching between the test PPG signal of the subject obtained in 410 and a plurality of pre-acquired PPG signals stored in a storage device (e.g., the storage device 220, the database 132). The storage device may also store a plurality of second parameters associated with the plurality of pre-acquired PPG signals. The second parameter module 330 may select at least one similar PPG signal from the plurality of pre-acquired PPG signals by matching the test PPG signal of the subject with the plurality of pre-acquired PPG signals, and determine the second parameter of the subject based at least on the second parameter associated with the at least one similar PPG signal.

In some embodiments, the second parameter module 330 may select a PPG signal from the pre-acquired PPG signals that is most similar to the test PPG signal as the at least one similar PPG signal, and designate the second parameter associated with the selected PPG signal as the second parameter of the test PPG signal.

In some embodiments, the second parameter module 330 may select multiple PPG signals from the pre-acquired PPG signals that are most similar to the test PPG signal as the at least one similar PPG signal based on rankings of the similarities of the pre-acquired PPG signals. The second parameter module 330 may then determine the second parameter of the test PPG signal based on second parameters associated with the selected PPG signals. For example, the second parameter of the test PPG signal may be a mean, a median, a weighted mean, a mode, etc., of the second parameters associated with the selected PPG signals. In some embodiments, the second parameter module 330 may determine the weight of a selected PPG signal based on the similarity of the selected PPG signal. Merely for example, a similarity parameter determined by the second parameter module 330 for indicating the similarity of the selected PPG signal may be used for determining the weight of the selected PPG signal. As another example, the second parameter module 330 may determine the weight of a selected PPG signal using the ranking of the selected PPG signal.

The second parameter module 330 may be configured to determine a similarity between a pre-acquired PPG signal and the test PPG signal using a predetermined matching strategy. In some embodiments, the second parameter module 330 may determine a difference (e.g., an l¹-distance, an l²-distance) between the test PPG signal and a pre-acquired PPG signals. The higher the difference, the lower the similarity. In some embodiments, the first parameters determined by the first parameter module 320 based on the test PPG signal may form a first feature vector (e.g., an M-dimensional feature vector). The second parameter module 330 may perform the matching based on the first feature vector. For example, the second parameter module 330 may determine a difference (e.g., an l¹-distance, an l²-distance) between the first feature vector of the test PPG signal and the first feature vector of a pre-acquired PPG signal. The higher the difference, the lower the similarity. The first feature vectors of the pre-acquired PPG signals may be pre-stored in the storage device, or be determined by the first parameter module 320 in real time. In some embodiments, the storage device may store the first feature vectors of the pre-acquired PPG signals instead of the pre-acquired PPG signals themselves.

Other matching strategies in the art may also be adopted by the second parameter module 330, and the above strategies are only for demonstration purposes and not intended to be limiting.

In some embodiments, the aforementioned pre-acquired PPG signals may serve as the standard PPG signals for training the model for the PTT determination. The second parameters associated with the pre-acquired PPG signals may be determined during the training of the model. Detailed descriptions of the training may be found elsewhere in the present disclosure (e.g., in connection with FIG. 8).

In some embodiments, the second parameter module 330 may determine a plurality of second parameters. Each of the second parameters is for determining a corresponding cardiovascular parameter. If the second parameter module 330 determines the second parameters of the test PPG signal by matching, correspondingly, the pre-acquired PPG signals may also be associated with a plurality of second parameters.

In 440, the determination module 350 may determine the PTT of the subject based at least on the plurality of first parameters and the second parameter via a trained model (e.g., the PTT model 153). The model may take the plurality of first parameters and the second parameter as at least part of its inputs and may determine a PTT (or any other cardiovascular parameter) as an output. By operating the model, the determination module 350 may determine a PTT for the subject.

In some embodiments, for determining the PTT, the first parameter module 320 may adopt a feature extracting manner other than the aforementioned group-feature extracting manner. For example, for each single-pulse PPG signal of the test PPG signal, the first parameter module 320 may determine a set of first parameters associated with the single pulse PPG signal. The determination module 350 may determine a PTT corresponding to the single-pulse PPG signal based on the set of first parameters associated with the single pulse PPG signal. Accordingly, the determination module 350 may determine a plurality of PTTs for the test PPG signal. The determination module 350 may determine a result PTT of the test PPG signal based on the plurality of PTTs as its output. For example, the result PTT may be a mean, a median, a weighted mean, a mode, etc., of the plurality of PTTs.

The above feature extracting manner may be referred to as an individual-specific feature extracting manner. When the PTT determination device 300 adopts such a feature extracting manner, a second parameter may be determined by the second parameter module 330 and be used for determining the plurality of PTTs of the test PPG signal.

In some embodiments, the determination module 350 may determine the PTT of the subject based further on one or more parameters associated with other factors involved in a PTT determination process, such as one(s) related to anthropometric character information of the subject (e.g., for enhancing the determination accuracy), and one(s) related to the performance of the sensor 120 (e.g., for reducing systematic error). In some embodiments, the determination module 350 may determine the PTT of the subject based further on one or more third parameters associated with the anthropometric character information of the subject. An exemplary process (process 600) for determining the PTT based further on the one or more third parameters is described in connection with FIG. 6. Features and embodiments of any operation of the process 500 may also be applied to a corresponding operation in the process 600.

In some embodiments, the determination module 350 may determine a plurality of cardiovascular parameters, each of at least some of which may be determined via a corresponding trained model based on a corresponding first parameter group determined in 420 and a corresponding second parameter determined in 430 according to the process 400.

In some embodiments, the determination module 350 may determine one or more second cardiovascular parameters based on a first cardiovascular parameter determined according to the process 400.

In some embodiments, the determination module 350 may determine a second cardiovascular parameter based on a plurality of first cardiovascular parameters determined according to the process 400.

It may be noted that the above descriptions of the process 400 are only for demonstration purposes, and not intended to limit the scope of the present disclosure. It is understandable that, after learning the major concept and the mechanism of the present disclosure, a person of ordinary skill in the art may alter the process 400 in an uncreative manner. For example, the operations above may be implemented in an order different from that illustrated in FIG. 4. For example, in some embodiments, the operations 430 may be performed before the operation 420 or the operation 410. One or more optional operations may be added to the flowcharts. One or more operations may be split or be combined. All such modifications are within the scope of the present disclosure.

FIG. 6 is a flowchart illustrating an exemplary process for determining a PTT based on a test PGG signal according to some embodiments of the present disclosure. Process 600 may be an example of the process 400, which further involves anthropometric characteristic information of the subject for the PTT determination. In some embodiments, one or more operations of process 600 illustrated in FIG. 6 may be implemented in the PTT determination device 300 (including the third parameter module 340) illustrated in FIG. 3. For example, the process 600 illustrated in FIG. 6 may be stored in a storage device (e.g., the storage device 220) in the form of instructions, and invoked and/or executed by at least one processor (e.g., the processor 210 of the computing device 200 as illustrated in FIG. 2).

In 610, the PPG signal module 310 may retrieve a test PPG signal of a subject. In 620, the first parameter module 320 may determine a plurality of first parameters related to the test PPG signal. The operations 610 and 620 may be the same as or similar to the operations 410 and 420, respectively, the descriptions of which are not repeated herein.

In 630, the third parameter module 340 may determine one or more third parameters based on the anthropometric characteristic information of the subject. The third parameter module 340 may determine the one or more third parameters by extracting features from the anthropometric characteristic information of the subject. The one or more third parameters may also serve as input(s) of the model (e.g., the PTT model 153) for determining PTT (or any other cardiovascular parameter).

In the present disclosure, features extracted form anthropometric characteristic information of a subject may be referred to as second features.

The third parameter module 340 may retrieve the anthropometric characteristic information of the subject from a storage device (e.g., the database 132, the storage device 220) or from one or more measuring devices (e.g., via the network 140). Alternatively or additionally, the third parameter module 340 may receive the anthropometric characteristic information via an input mean provided by the PTT determination device 300 for a user (e.g., the subject).

In some embodiments, the third parameter module 340 may retrieve at least one second feature extracting mean and extract the second features based on the at least one second feature extracting mean. The second feature extracting mean may also be in the form of, for example, a look-up table, a feature extracting model, or the like, or a combination thereof. The third parameter module 340 may retrieve the at least one second feature extracting mean from a storage device (e.g., the storage device 220) or the server 130.

In some embodiments, the second feature extracting mean may include a look-up table including a plurality of items, each of which represents a second feature to be extracted. The third parameter module 340 may include models or functions for performing the feature extraction. Alternatively or additionally, the third parameter module 340 may retrieve the models or functions from a storage device (e.g., the storage device 220) or the server 130.

In some embodiments, the second feature extracting mean may include a feature extracting model. By operating the feature extracting model on the anthropometric characteristic information of the subject anthropometric characteristic information of the subject, the third parameter module 340 may extract one or more corresponding second features thereby obtain one or more corresponding third parameters.

In some embodiments, second features to be extracted by the third parameter module 340 may include but not limited to: the square of the height (height²) of the subject, the body mass index (BMI, BMI=weigh/height²) of the subject, etc. It may be noted that, the above second features are only provided for demonstration purposes and not intended to be limiting.

The third parameter module 340 may extract N second features (N is a positive integer larger than 1) from the anthropometric characteristic information of the subject. Correspondingly, the third parameter module 340 may determine N third parameter(s). In some embodiments, the PTT determination device 300 may determine T=M+N+1 parameters in total (including the plurality of first parameters, the second parameter, and the third parameter(s)). In some embodiments, a value range of T may be [30, 150]. In some specific embodiments, a value range of T may be [40, 80]. In some more specific embodiments, a value of T may be about 70 (e.g., 69, 70, 71).

In some embodiments, the second feature extracting mean and the first feature extracting mean may be integrated into a comprehensive feature extracting mean. For example, the comprehensive feature extracting mean may include a look-up table recording both the first features and the second features to be extracted. As another example, the comprehensive feature extracting mean may take the test PPG signal and the anthropometric characteristic information of the subject as its inputs and determine the plurality of first parameters and the one or more third parameters as its outputs. Correspondingly, the first parameter module 320 and the third parameter module 340 may be integrated into a single module.

In some embodiments, the second feature extracting mean or the comprehensive feature extracting mean may be generated during the training of the model for determining the PTT (e.g., the PTT model 153), the descriptions of which may be found elsewhere in the present disclosure (e.g., in connection with FIG. 8).

In 640, the second parameter module 330 may determine a second parameter of the subject, the second parameter indicating a random effect of the subject. In some embodiments, the operation 640 may be the same as or similar to the operation 430, the descriptions of which are not repeated herein. In some embodiments, the operation 640 may be a modified version of the operation 430 considering the one or more third parameters determined in the operation 620, the descriptions of which are provided as following.

In some embodiments, the second parameter may be predetermined (e.g., by the second parameter module 330) based on a pre-acquired test PPG signal of the subject, the PTT of the subject, and the anthropometric characteristic information of the subject. The first parameter module 320 may determine a plurality of first parameters by extracting first features from the pre-acquired test PPG signal, and the third parameter module 340 may determine one or more third parameters by extracting second features from the anthropometric characteristic information of the subject. The second parameter module 330 may then determine the second parameter of the subject based on the PTT of the subject, the plurality of first parameters, the one or more third parameters, and the model for determining the PTT (which also takes the one or more third parameters as it inputs) by fitting.

In some embodiments, the second parameter module 330 may determine the second parameter of the subject in real-time by performing a matching. The matching may be based on a similarity of a pre-acquired PPG signal with respect to the test PPG signal and a similarity of the anthropometric characteristic information of the subject associated with the pre-acquired PPG signal with respect to that of the subject of the test PPG signal. In some embodiments, the second parameter module 330 may determine a first difference (e.g., a Euclidean distance) between the test PPG signal and a pre-acquired PPG signal and a second difference between the anthropometric characteristic information of the subject of the test PPG signal and that of the subject associated with the pre-acquired PPG signal. The second parameter module 330 may further determine a difference indicator based on the first difference and the second difference (e.g., a sum, a weighted sum, a mean, a weighted mean). The higher the difference indicator, the lower the similarity. In some embodiments, the first parameters and the third parameter(s) determined in the operations 620 and 630 may form a second feature vector (e.g., an (M+N)-dimensional feature vector). The second parameter module 330 may perform the matching based on the second feature vector. For example, the second parameter module 330 may determine a difference (e.g., an l¹-distance, an l²-distance) between the second feature vector of the test PPG signal and the second feature vector of a pre-acquired PPG signal. The higher the difference, the lower the similarity. The second feature vectors of the pre-acquired PPG signals may be pre-stored in the storage device, or be determined by the first parameter module 320 and the third parameter module 340 in real time. In some embodiments, the storage device may store the second feature vectors of the pre-acquired PPG signals instead of the pre-acquired PPG signals themselves.

In some embodiments, the aforementioned pre-acquired PPG signals may serve as the standard PPG signals for training the model for the PTT determination. The second parameters associated with the pre-acquired PPG signals may be determined during the training of the model. Detailed descriptions of the training may be found elsewhere in the present disclosure (e.g., in connection with FIG. 8).

In 650, the determination module 350 may determine the PTT of the subject based at least on the plurality of first parameters, the second parameter, and the one or more third parameter via a trained model (e.g., the PTT model 153). The model may take the plurality of first parameters, the second parameter, and the one or more third parameters as at least part of its inputs and may determine a PTT (or any other cardiovascular parameter) as an output. By operating the model, the determination module 350 may determine a PTT for the subject. The operation 650 may be similar to the operation 440, the descriptions of which are not repeated herein.

It may be noted that the above descriptions of the process 600 are only for demonstration purposes, and not intended to limit the scope of the present disclosure. It is understandable that, after learning the major concept and the mechanism of the present disclosure, a person of ordinary skill in the art may alter the process 600 in an uncreative manner. For example, the operations above may be implemented in an order different from that illustrated in FIG. 4. For example, in some embodiments, the operations 640 may be performed before the operation 630, 620, or 610. One or more optional operations may be added to the flowcharts. One or more operations may be split or be combined. All such modifications are within the scope of the present disclosure.

FIG. 7 is a schematic diagram illustrating an exemplary model training module according to some embodiments of the present disclosure. Model training module 700 is an example of the mobile training module 131 (as illustrated in FIG. 1), which may be configured to train a model (e.g., PTT model 153) for determining the PTT (and/or any other cardiovascular parameter) of a subject based on the PPG signal of the subject. The model training module 700 may include a candidate feature unit 710, a training dataset unit 720, a feature selection unit 730, a model training unit 740, and a feature extracting mean unit 750. In some embodiments, the model training module 700 may further include a model test unit 760.

The model training module 700 and the modules thereof may be implemented by the computing device 200 illustrated by FIG. 2.

The candidate feature unit 710 may be configured to determine a first plurality of candidate features. The first plurality of candidate features may include candidate features associated with at least one of a PPG signal, a first-order derivative of the PPG signal, and a second-order derivative of the PPG signal. In some embodiments, the first plurality of candidate features may also include candidate features associated with the anthropometric character information of the subject.

The training dataset unit 720 may be configured to obtain a training dataset including a plurality of standard PPG signals and a plurality of standard PTTs (or any other cardiovascular parameter) corresponding to the standard PPG signals. In some embodiments, the training dataset may further include the anthropometric character information of the subject associated with each of the standard PPG signals thereof.

The feature selection unit 730 may be configured to select, based on the training dataset, a second plurality of candidate features from the first plurality of candidate features via a feature selection routine. The feature selection unit 730 may perform the feature selection routine on the first plurality of candidate features to remove redundant or irrelevant features from the first plurality of candidate features, thereby obtain the second plurality of candidate features.

The model training unit 740 may be configured to train a model (e.g., the PTT model 153) for PTT (or any other cardiovascular parameter) determination by: determining a weight associated with each of the second plurality of candidate features by solving, based on the training dataset, a regression function related to the second plurality of candidate features; selecting, based on the determined weights, a plurality of target features form the second plurality of candidate features; and generating the model for the PTT determination based on the plurality of target features and the weights thereof as the trained model.

The feature extracting mean unit 750 may be configured to generate at least one feature extracting mean according to the target features. The at least one feature extracting mean may include, for example, a look-up table and/or a feature extracting model. The at least one feature extracting mean may be transmitted to or be retrieved by the first parameter module 320 and/or the third parameter module 340 (optional) for determining a plurality of first parameters and/or one or more third parameters for the PTT determination.

The model training module 700 may train the model for determining the PTT (or any other cardiovascular parameter) via a process (e.g., process 800) described in connection with FIG. 8 or a process (e.g., process 900) described in connection with FIG. 9.

The model test unit 760 may be configured to test the performance of the trained model. When the trained model fails such a test, the model test unit 760 may trigger the retraining of the mode.

It may be noted that, the above descriptions about the model training module 700 are only for illustration purposes, and are not intended to limit the present disclosure. It is understandable that, after learning the major concept and the mechanism of the present disclosure, a person of ordinary skill in the art may alter the model training module 700 in an uncreative manner. The alteration may include combining and/or splitting modules or sub-modules, adding or removing optional modules or sub-modules, etc. For example, the feature selection unit 730 may be removed from the model training module 700. All such modifications are within the scope of the present disclosure.

FIG. 8 is a flowchart illustrating an exemplary process for training a model for the PTT determination according to some embodiments of the present disclosure. Process 800 may be performed to train a model for determining the PTT (or any other cardiovascular parameter) of a subject based on a test PPG signal of the subject. In some embodiments, one or more operations of process 400 illustrated in FIG. 4 may be implemented in the model training module 700 illustrated in FIG. 7 (or the server 130 illustrated in FIG. 1). For example, the process 800 illustrated in FIG. 8 may be stored in a storage device (e.g., the storage device 220) in the form of instructions, and invoked and/or executed by at least one processor (e.g., the processor 210 of the computing device 200 as illustrated in FIG. 2).

In 810, the candidate feature unit 710 may determine a first plurality of candidate features. The first plurality of candidate features may include features associated with at least one of a PPG signal, a first-order derivative of the PPG signal, and a second-order derivative of the PPG signal. In some embodiments, the candidate feature unit 710 may determine features associated with one or more feature sources including a single-pulse PPG signal, the first derivative of the single-pulse PPG signal, and the second derivate of the single-pulse PPG signal.

For example, the candidate feature unit 710 may extensively determine possible feature points on the one or more feature sources, and extensively determine possible features using the attributes of the determined feature points.

For example, one or more candidate features may be an attribute of a certain feature point included in a certain feature source such as, the intensity value of the first trough/peak of a single-pulse PPG signal, the intensity value of the first trough/peak of the of the first-order derivative of the single-pulse PPG signal, the intensity value of the second trough/peak of the second-order derivative of the single-pulse PPG signal, the timestamp (with respect to the starting point of the whole test PPG signal or with respect to the starting point of the current single-pulse PPG signal) of the second zero-crossings of the first/second-order derivative of the single-pulse PPG signal, the intensity value of a point in the single-pulse PPG signal corresponding to the first trough/peak of the first/second-order derivative of the single-pulse PPG signal, etc.

As another example, one or more candidate features may be based on attributes of multiple feature points of a same feature source, such as a ratio of the intensity value of the first peak to that of the second peak in the single-pulse PPG signal, a difference of the intensity value of the second peak and that of the second trough in the first-order derivative, a sum of the intensity values of the first peak, the second peak, and the third peak of the second-derivative, a time interval between the third zero-crossing and the fourth zero-crossing of the first-order derivative, a time interval between the first peak and the second trough of the single-pulse PPG signal, etc.

As a further example, one or more candidate features may be based on attributes of multiple feature points of different feature sources, such as a ratio of the intensity value of the second trough in the single-pulse PPG signal to that of the second peak of the second derivative, a time interval between the first peak of the single-pulse PPG signal and the third zero-crossing of the second derivative.

In some embodiments, the first plurality of candidate features may also include candidate features associated with the anthropometric character information of the subject. For example, one or more candidate features may be based on one or more anthropometric characteristic parameters, such as height, age, weight, sex (e.g., 1 for male and 0 for female), body fat percentage, etc. Exemplary candidate features associated with the anthropometric character information of the subject may include height, age, weight, sex, the square of height, the cube of height, the BMI, etc.

The candidate feature unit 710 may determine F candidate features as the first plurality of candidate features. Merely for demonstration purpose, the value range of F may be [500, 1000]. In some specific embodiments, the value of F may be 700.

In 820, the training dataset unit 720 may obtain a training dataset including a plurality of standard PPG signals and a plurality of standard PTTs corresponding to the standard PPG signals. For example, the training dataset unit 720 may retrieve the plurality of standard PPG signals and the plurality of PTT from a storage device (e.g., the storage device 220, the database 132). For each standard PPG signal in the training dataset, the corresponding standard PTT may server as the supervisory output (or label) of the standard PPG signal.

In some embodiments, the training dataset unit 720 may further retrieve, from the storage device, the anthropometric characteristic information of a subject associated with each of the standard PPG signals. The training dataset may further include the anthropometric characteristic information. The anthropometric characteristic information of a subject may be associated with a standard PPG signal of the same subject in the training dataset, and the corresponding standard PTT may serve as the supervisory output (or label) of the standard PPG signal and the anthropometric characteristic information.

In some embodiments, the database 132 may be configured or organized as a superior training dataset. The training dataset unit 720 may retrieve a sub-dataset of the database 132 as a training dataset of the model. Merely for example, the training dataset may include 70% training data of the database 132, and the other 30% training data of the database 132 may be used for testing the stability of the trained model. In some embodiments, the training dataset may randomly retrieve the training data of the database 132 to construct or form the training dataset of the model.

In 830, the feature selection unit 730 may select, based on the training dataset, a second plurality of candidate features from the first plurality of standard PPG signals using a feature selection routine. Exemplary feature selection routines may include wrapper based routine, filter based routine, and embedded based routine.

In some embodiments, the feature selection unit 730 may perform a correlation-based feature selection (CFS) routine to select the second plurality of candidate features from the first plurality of candidate features. Via the CFS routine, the feature selection unit 730 may determine, based on the training dataset, a plurality of correlations between the first plurality of candidate features, and select the second plurality of candidate features based on the plurality of correlations. The feature selection unit 730 may determine (or measure) the correlation between any two of the first plurality of candidate features to obtain the plurality of correlations. The feature selection unit 730 may use various metrics in the art for measuring the correlations, such as Pearson's correlation coefficient, Spearman's rank correlation coefficient, minimum description length (MDL), symmetrical uncertainty, relief, or the like, or a combination thereof. In some embodiments, the feature selection unit 730 may generate, based on the training dataset or at least a part of it, a covariance matrix of the first plurality of candidate features serving as a metric for measuring correlations of the first plurality of candidate features. Using the covariance matrix, the feature selection unit 730 may solve a target function associated with the CFS routine, and thereby select the second plurality of candidate features from the first plurality of candidate features. Merely for example, via the CFS routine, the feature selection unit 730 may select 20%-50% of the candidate features of the first plurality of candidate features as the second plurality of candidate features. In some specific embodiments, the number of the candidate features of the first plurality of candidate features may be about 700, and the number of the candidate features of the second plurality of candidate features may be about 150-200.

In operations 840, 850, and 860, the model training unit 740 may train a model for PTT determination based on the training data.

In 840, the model training unit 740 may determine a weight associated with each of the second plurality of candidate features by solving, based on the training dataset, a regression function. The model training unit 740 may construct the regression function with respect to the second plurality of candidate features and a second parameter of a corresponding subject. For example, the regression function may include at least one variable associated with the second plurality of candidate features and at least one variable associated with the second parameter.

By solving the regression function, a weight associated with each of the second plurality of candidate features (or a coefficient associated with a corresponding variable of the preliminary model) may be determined by the model training unit 740. Meanwhile, a second parameter may also be determined for each of the standard PPG signals, which may indicate the random effect of the subject associated with the standard PPG signal.

The second parameter determined by the model training unit 740 may be stored in the database 132 (or another storage device such as the storage device 220). The stored second parameter may be associated with the corresponding standard PPG signal in the database 132. In some embodiments, one or more of the stored second parameters may be retrieved by the second parameter module 330 for determining a second parameter of a subject in an aforementioned PTT determination process (e.g., the process 400 or 600). For example, when a pre-acquired PPG signal of the subject was used as a standard PPG signal for training the model, and identity information of subjects associated with each of the standard PPG signals is also included in the database 132, the second parameter module 330 may directly retrieve the second parameter of the subject from the database 132. As another example, the second parameter module 330 may perform a match between the test PPG signal and the standard PPG signals included in the database 123 and retrieve one or more second parameters based on the matching result.

In some embodiments, the second parameters of the standard PPG signals may be configured to satisfy a certain distribution, such as a normal distribution, a generalized normal distribution. Such a distribution may serve as a restriction of the regression function. The model training unit 740 may solve the regression function using various approaches in the art, such as an expectation-maximization (EM) based approach.

In some embodiments, the regression function may be a least absolute shrinkage and selection operator (LASSO) based regression function. By solving the LASSO based regression function, weights associated with some of the second plurality of candidate features may be set to be zero. For example, the LASSO based regression function may be in the form of Equation (1), which may be expressed as:

min{∥y−Xβ−α∥₂²+λ₁∥β∥₁}, s.t. α˜N(0,σ²), Equation (1)

wherein y refers to a standard PTT (or any other cardiovascular parameter), X refers to the second plurality of candidate features extracted from the standard PTT, α refers to a second parameter of the subject associated with the standard PTT, β refers to the weights associated with the second plurality of candidate features, and function N(0,σ²) represents a normal distribution function with a mean as zero and a standard deviation as a, and A may be a predetermined parameter that determines the amount of regularization. The model training unit 740 may solve the Equation (1) based on the training dataset using, for example, an EM based approach, thereby determine the weights associated with the second plurality of candidate features (some of the weights may be set as zeroes), a second parameter for each standard PTT in the training dataset, and the standard deviation a.

When the model training unit 740 uses a regression function purposely causing some of the weights set as zeroes such as LASSO based regression function, the operation 840 may also be viewed as an embedded feature selection operation, which may select features and train the model at the same time.

In 850, the model training unit 740 may select, based on the determined weights, a plurality of target features from the second plurality of candidate features.

In some embodiments, the model training unit 740 may select features whose weight is not zero as the plurality of target features, especially when a regression function purposely causing some of the weights set as zeroes is used for determining the weights.

In some embodiments, the model training unit 740 may select, or designate, all the features of the second plurality of candidate features as the plurality of target features, for example, when the weights of the second plurality of the candidate features are all determined to be non-zeroes.

In some embodiments, the model training unit 740 may select the plurality of target features based on rankings of the absolute value of the determined weights (a higher absolute value may lead to a higher ranking). The model training unit 740 may select the candidate features whose rankings are above a certain ranking as the plurality of target features.

In some embodiments, the model training unit 740 may select candidate features whose absolute value is above a predetermine threshold as the plurality of target features.

Merely for example, when a Lasso based regression function is used for training the model, the model training unit 740 may select 25%-50% of the candidate features of the second plurality of candidate features as the plurality of target features. In some specific embodiments, the number of the features of the first plurality of candidate features may be about 700, and the number of the features of the plurality of target features may be about 50-100. In some more specific embodiments, the number of the features of the plurality of target features may be about 70 (e.g., 69, 70, 71).

In 860, the model training unit 740 may generate the model for the PTT determination based on the plurality of target features and the weights thereof. The generated model may be the trained model for determining the PTT of the subject in the aforementioned PTT determination processes (e.g., the processes 400 and 600).

In some embodiments, the generated model may be in the form of a linear regression function. The model may include variables corresponding to the plurality of target features, and the coefficient of each of the variables may be set (e.g., by the model training unit 740) as the corresponding weight.

In some embodiments, in 860, to generate the model, the model training unit 740 may re-train the model formed by the plurality of target features with initial weights set as the corresponding weights determined in 840. For example, the model training unit 740 may re-train the model (optionally) when candidate features with non-zero weights are excluded from the plurality of target features. When the re-training is completed, the model training unit 740 may further remove features with weights equal to zero (if any) from the plurality of target features. The regression function used for the re-training may be similar to or different from the regression function used in the first training.

In 870, the feature extracting mean unit 750 may generate at least one feature extracting mean according to the plurality of target features. Based on the obtained training dataset and the results of the operations 830, 840, 850 (and 860 in some embodiments), the plurality of target features may only include aforementioned first features or include both of the first features and the aforementioned second feature(s). Correspondingly, the at least one feature extracting mean may include the at least one first feature extracting mean and/or the at least one second feature extracting mean for determining the plurality of first parameters and/or the one or more third parameters in the aforementioned PTT determination processes (e.g., the processes 400 and/or 600). For example, the at least one feature extracting mean generated by the feature extracting mean unit 750 may include a look-up table recording the plurality of target features (including the first features and/or the second features). As another example, the at least one feature extracting mean generated by the feature extracting mean unit 750 may include a feature extracting model for extracting the plurality of target features from a test PPG signal for the determination of PTT (or any other cardiovascular parameter).

In some embodiments, the at least one feature extracting mean generated by the feature extracting mean unit 750 may be a comprehensive feature extracting mean integrate the first feature extracting mean(s) and or the second feature extracting mean(s).

In some embodiments, the process 800 may further include an operation (optional) for testing the performance of the trained model, which may be performed by the model test unit 760. In 820, the training dataset unit 720 may retrieve a first sub-dataset of a superior training dataset (e.g., the database 132) as the training dataset of the model. The model test unit 760 may retrieve a second sub-dataset from the superior training dataset as a test dataset. Merely for example, the training dataset may include 70% training data of the superior training dataset, and the test dataset may include the other 30% training data of the superior training dataset (the above ratios may be adjusted in need). After a trained model is obtained in the operation 860, the model test unit 760 may test the performance (e.g., the accuracy and the stability) of the trained model with the test dataset. It the trained model fails such a test, the model test unit 760 may trigger a re-determination (or re-allocation) of the training dataset and the test dataset based on the superior training dataset (e.g., in a ratio of 70:30 or any other proper ratio). The operations 830 to 860, or the operations 840 to 860 may be performed again to train a model based on the newly determined training dataset. The model test unit 760 may test the performance of the model with the newly determined test dataset.

For testing the performance of the trained model, for each of the standard PPG signals in the test dataset, the model test unit 760 may extract first features and/or second feature(s) from the standard PPG signals and/or the corresponding anthropometric characteristic information (e.g., via the first feature extracting mean(s) and/or the second feature extracting mean(s) determined by the feature extracting mean unit 750); and operate the trained model using the extracted first features and/or second feature(s) and the second parameter of the standard PPG signal (e.g., determined in the operation 840) to obtain a predicted PTT of the standard PPG signal. The model test unit 760 may determine the performance of the trained model based on each standard PTT in the test dataset and the corresponding predicted PTT.

In some embodiments, to test the accuracy of the trained model, the model test unit 760 may compare a distribution of the predicted PTTs and a distribution of the standard PTTs, determine a mean and/or a variance of the residuals (a residual is a difference between a standard PTT and a corresponding predicted PTT), and/or determine a distribution of the residuals.

In some embodiments, to test the stability of the trained model, the model test unit 760 may perform a test cycle for a plurality of times (e.g., 10 times, 20 times, 30 times). At each test cycle, the model test unit 760 may randomly determine a test dataset from the superior training dataset and test the accuracy of the trained model with the determined test dataset. When the accuracy of the trained model keeps at a high level over at least most of the test cycles (e.g., 80%, 90%, 95%, 100%), the trained model may pass the stability test. Otherwise, the trained model may fail the stability test.

In some embodiments, the operation 830 may be removed from the process 800. A regression function (e.g., a LASSO based regression function) may be constructed with respect to the first plurality of candidate features, and a trained model may be obtained by solving the regression function.

In some embodiments, the PTT determination device 110 may retrieve the model trained by the server 130 via the network 140. Alternatively or additionally, the trained model may be inputted into a storage device (e.g., the storage device 220) of the PTT determination device 110 during the manufacture of the PTT determination device 110.

In some embodiments, after the PTT determination device 110 obtains the trained model, the PTT determination device 110 may adjust the weights (or coefficients) of the model based on one or more test PPG signals of a user and the corresponding PTT(s) (e.g., determine via a PTT measurement or determination routine in the art) of the user, so as to generate a user-specific model with improved accuracy with respect to the user. In some embodiments, the PTT determination device 110 may generate one or more user-specific PTT determination models based on the trained model for one or more users of the PTT determination device 110.

It may be noted that the above descriptions of the process 800 are only for demonstration purposes, and not intended to limit the scope of the present disclosure. It is understandable that, after learning the major concept and the mechanism of the present disclosure, a person of ordinary skill in the art may alter the process 800 in an uncreative manner. For example, the operations above may be implemented in an order different from that illustrated in FIG. 8. One or more optional operations may be added to the flowcharts. One or more operations may be split or be combined. All such modifications are within the scope of the present disclosure.

Having thus described the basic concepts, it may be rather apparent to those skilled in the art after reading this detailed disclosure that the foregoing detailed disclosure may be intended to be presented by way of example only and may be not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment may be included in at least one embodiment of the present disclosure. Therefore, it may be emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that may be not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, may be not intended to limit the claimed processes and methods to any order except as may be specified in the claims. Although the above disclosure discusses through various examples what may be currently considered to be a variety of useful embodiments of the disclosure, it may be to be understood that such detail may be solely for that purposes, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purposes of streamlining the disclosure aiding in the understanding of one or more of the various inventive embodiments. This method of disclosure, however, may be not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein may be hereby incorporated herein by this reference in its entirety for all purposes, excepting any prosecution file history associated with same, any of same that may be inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and describe.

1. A system for determining a cardiovascular parameter related to a cardiovascular system of a subject, comprising at least one processor and at least one storage device for storing instructions that when executed by the at least one processor, cause the system to:
retrieve a photoplethysmogram (PPG) signal of a subject;
determine a plurality of first parameters related to the PPG signal;
determine a second parameter of the subject, the second parameter indicating a random effect of the subject; and
determine the cardiovascular parameter based at least on the plurality of first parameters and the second parameter via a trained model.

2. The system of claim 1, wherein to determine the second parameter of the subject, the system is caused to:
select, from a plurality of pre-acquired PPG signals, at least one similar PPG signal by matching the PPG signal of the subject with the plurality of pre-acquired PPG signals, wherein each of the plurality of pre-acquired PPG signals is associated with a signal parameter; and
determine the second parameter of the subject based at least on a signal parameter associated with the at least one similar PPG signal.

3. The system of claim 2, wherein a plurality of signal parameters associated with the plurality of pre-acquired PPG signals satisfy a normal distribution or a generalized normal distribution.

4. The system of claim 1, wherein to determine the plurality of first parameters, the system is caused to:
retrieve at least one feature extracting mean; and
determine at least some of the plurality of first parameters by extracting, via the at least one feature extracting mean, features based on at least one of the PPG signal, a first-order derivative of the PPG signal, or a second-order derivative of the PPG signal.

5. The system of claim 4, wherein
the system is caused further to train the model, and to train the model, the system is caused to:
determine a first plurality of candidate features, the first plurality of candidate features including features associated with at least one of a PPG signal, a first-order derivative of the PPG signal, or a second-order derivative of the PPG signal;
obtain a training dataset, the training dataset including a plurality of standard PPG signals and a plurality of standard cardiovascular parameters corresponding to the plurality of standard PPG signals;
select, based on the training dataset, a second plurality of candidate features from the first plurality of candidate features using a feature selection routine;
determine a weight associated with each of the second plurality of candidate features by solving, based on the training dataset, a regression function, wherein:
the regression function includes at least one variable associated with the second plurality of candidate features and at least one variable associated with the second parameter; and
by solving the regression function, a sample second parameter is determined for each of the plurality of standard PPG signals;
select, based on the determined weights, a plurality of target features from the second plurality of candidate features; and
generate the model based on the plurality of target features and the weights thereof, wherein the model includes a variable associated with the second parameter; and
to retrieve the at least one feature extracting mean, the system is caused to:
generate the at least one feature extracting mean according to the plurality of target features.

6. The system of claim 5, wherein to select the second plurality of candidate features from the first plurality of candidate features, the system is caused to:
determine, based on the training dataset, a plurality of correlations between the first plurality of candidate features, wherein the second plurality of candidate features are selected based on the plurality of correlations.

7. The system of claim 5, wherein by solving the regression function based on the training dataset, one or more of the weights are set to be zero.

8. The system of claim 5, wherein the determined sample second parameters of the plurality of standard PPG signals satisfy a normal distribution or a generalized normal distribution.

9. The system of claim 5, wherein the regression function is solved using an expectation maximization algorithm.

10. The system of claim 5, wherein a count of the first plurality of candidate features ranges between 500 and 1000.

11. The system of claim 1, wherein:
the model further includes one or more variables associated with anthropometric characteristic information of the subject;
the system is caused further to determine, based on the anthropometric characteristic information of the subject, one or more third parameters of the subject; and
the cardiovascular parameter is determined based further on the one or more third parameters of the subject.

12. The system of claim 1, further comprising:
a sensor, configured to generate a raw PPG signal of the subject by detecting pulses of the subject for a predetermined time, wherein the system is caused further to generate the PPG signal by preprocessing the raw PPG signal.

13. The system of claim 1, wherein a count of the plurality of first parameters ranges between 30 and 150.

14-26. (canceled)

27. A method for determining a cardiovascular parameter related to a cardiovascular system of a subject, implemented on at least one device that has at least one processor and a storage device, the method comprising:
retrieving, by the at least one processor, a photoplethysmogram (PPG) signal of a subject;
determining, by the at least one processor, a plurality of first parameters related to the PPG signal;
determining, by the at least one processor, a second parameter of the subject, the second parameter indicating a random effect of the subject; and
determining, by the at least one processor, the cardiovascular parameter based at least on the plurality of first parameters and the second parameter via a trained model.

28. The method of claim 27, further comprising:
selecting, from a plurality of pre-acquired PPG signals, at least one similar PPG signal by matching the PPG signal of the subject with the plurality of pre-acquired PPG signals, wherein each of the plurality of pre-acquired PPG signals is associated with a signal parameter; and
determining the second parameter of the subject based at least on a signal parameter associated with the at least one similar PPG signal.

29. (canceled)

30. The method of claim 27, wherein the determining a plurality of first parameters comprises:
retrieving at least one feature extracting mean; and
determining at least some of the plurality of first parameters by extracting, via the at least one feature extracting mean, features based on at least one of the PPG signal, a first-order derivative of the PPG signal, or a second-order derivative of the PPG signal.

31. The method of claim 30, further comprising:
training the model by:
determining a first plurality of candidate features, the first plurality of candidate features including features associated with at least one of a PPG signal, a first-order derivative of the PPG signal, Or a second-order derivative of the PPG signal;
obtaining a training dataset, the training dataset including a plurality of standard PPG signals and a plurality of standard cardiovascular parameters corresponding to the plurality of standard PPG signals;
selecting, based on the training dataset, a second plurality of candidate features from the first plurality of candidate features using a feature selection routine;
determining a weight associated with each of the second plurality of candidate features by solving, based on the training dataset, a regression function, wherein:
the regression function includes at least one variable associated with the second plurality of candidate features and at least one variable associated with the second parameter; and
by solving the regression function, a sample second parameter is determined for each of the plurality of standard PPG signals;
selecting, based on the determined weights, a plurality of target features from the second plurality of candidate features; and
generating the model based on the plurality of target features and the weights thereof, wherein the model includes a variable associated with the second parameter; and
retrieving the at least one feature extracting mean by:
generating the at least one feature extracting mean according to the plurality of target features.

32-36. (canceled)

37. The method of claim 27, wherein:
the model further includes one or more variables associated with anthropometric characteristic information of the subject;
the method further comprises determining, based on the anthropometric characteristic information of the subject, one or more third parameters of the subject; and
the cardiovascular parameter is determined based further on the one or more third parameters of the subject.

38. The method of claim 27, further comprising:
generating, by a sensor, a raw PPG signal of the subject by detecting pulses of the subject for a predetermined time; and
generating the PPG signal by preprocessing the raw PPG signal.

39. (canceled)

40. A non-transitory computer readable medium, storing instructions, the instructions, when executed by a processor, causing the processor to execute operations comprising:
retrieving a photoplethysmogram (PPG) signal of a subject;
determining a plurality of first parameters related to the PPG signal;
determining a second parameter of the subject, the second parameter indicating a random effect of the subject; and
determining the cardiovascular parameter based at least on the plurality of first parameters and the second parameter via a trained model.
